(19) United States
(12) Patent Application Publication
(10) Pub. No.: US 2019/0209783 A1
(43) Pub. Date: Jul. 11, 2019

(54) Fluid Discharge Device, Method for Producing and Method for Operating a Fluid Discharge Device for Discharging a Fluid

(71) Applicant: Robert Bosch GmbH, Stuttgart (DE)

(72) Inventors: Fabian Utermoehlen, Lippstadt (DE); Andreas Merz, Freiberg Am Neckar (DE)

(21) Appl. No.: 16/301,015

(22) PCT Filed: May 19, 2017
(86) PCT No.: PCT/EP2017/062082

§ 371 Date: Nov. 13, 2018

(30) Foreign Application Priority Data
May 19, 2016 (DE) 10 2016 208 622.7

(51) Int. Cl. A61M 5/315; A61M 5/24

(52) U.S. Cl. CPC A61M 5/31551; A61M 5/3155; A61M 2005/3126; A61M 2205/332; A61M 5/2455

(57) ABSTRACT

A fluid discharge device for discharging a fluid has at least one release device for releasing the fluid from a fluid storage container. In addition, the fluid discharge device has a spring, which is tensioned between the release device and a force sensor, and which is designed to exert a restoring force on the force sensor with the releasing of the fluid. Finally, the fluid discharge device has the force sensor, which is designed to detect the restoring force acting on the force sensor with the releasing of the fluid.

### PRIOR ART

The approach starts from a device or a method of the type according to the independent claims. The current approach also relates to a computer program.

A deficiency of endogenous insulin in diabetes mellitus is treated by injection of an insulin preparation. Customary injection devices are disposable syringes, permanent insulin pumps, and disposable or multi-use pens. To have an overview of the quantities of insulin already injected, it can be helpful for diabetes patients if at least a last injected insulin quantity is stored.

WO 2015/074979 A2 describes an insulin pen in which a patient, when setting the quantity of insulin to be administered, has to turn a dosing knob, which executes both a rotation movement and a translation movement.

### DISCLOSURE OF THE INVENTION

Against this background, the approach presented here concerns a fluid discharge device, a method for producing and a method for operating a fluid discharge device for discharging a fluid, and, finally, a corresponding computer program, in accordance with the main claims. Advantageous developments and improvements of the fluid discharge device set out in the independent claim are possible by means of the measures set out in the dependent claims.

A fluid discharge device for discharging a fluid is proposed. The fluid discharge device has at least one release device for releasing the fluid from a fluid storage container. The fluid discharge device moreover has a spring, which is tensioned between the release device and a force sensor, and which is configured to exert a restoring force on the force sensor upon release of the fluid. Lastly, the fluid discharge device has the force sensor, which is configured to sense the restoring force acting on the force sensor upon release of the fluid.

The advantages that can be achieved by the current approach are that a quantity of the fluid released by the fluid discharge device can be quickly determined by means of the restoring force sensed by the force sensor upon release of the fluid. The restoring force of a spring can be calculated, for example, by the formula F′=−D×s. F′ is the restoring force, D is a spring constant, and s is a length of the spring. The restoring force is accordingly dependent, for example, on the length and the spring constants of the spring. The spring constant mainly depends, for example, on the material of the spring and its geometry, i.e. its wire diameter, spring diameter and number of turns.

For patients who self-administer medicaments by means of medicament discharge devices such as insulin pens, a knowledge of the released quantity of medicament may be important since, for example, many diabetes patients require different quantities of insulin several times over the course of a day. If a previously administered quantity of insulin is displayed to the patient, this can make the further course of treatment easier for the patient.

For this purpose, it is of advantage if the fluid discharge device according to one embodiment has a determination device which, using the sensed restoring force, is configured to determine and/or store the released fluid quantity. To assign a time to the released fluid quantity, the fluid discharge device can have a time detection device for detecting at least one time point of the released fluid quantity, wherein the time point of the released fluid quantity can likewise be stored.

To display the released fluid quantity and/or at least one stored fluid quantity and/or a time point of the stored fluid quantity, the fluid discharge device can advantageously have a display device for displaying the released fluid quantity and/or at least one stored fluid quantity and/or a time point of the stored fluid quantity. The display device can have an e-ink display, i.e. an electronic paper display, which permits a permanent display using very low energy.

It is also of advantage if the fluid discharge device according to one embodiment comprises a communication interface which is configured to make the released fluid quantity available to an appliance arranged externally with respect to the fluid discharge device. The communication interface can make the released fluid quantity available wirelessly, such that the released fluid quantity can be read off by the patient, for example by means of a cell phone, such as a smart phone, or a computer and is thus available at all times to the patient.

The force sensor can be quartz-based and/or piezo-based and/or MEMS-based, in order to sense, on a small surface area, a reliable measured value in the form of the restoring force.

The release device can have a setting element for setting a fluid quantity, and a housing element, wherein the setting element is inserted at least partially into the housing element upon release of the fluid. When the patient is setting the released fluid quantity, he or she can screw the setting element into an inner thread of the housing element, for example via an outer thread of the setting element, or can unscrew it from the inner thread of the housing element. After completion of the setting of the fluid quantity and locking of the rotation movement, the set fluid quantity to be released can be released by the patient applying pressure to the pressure sensor, wherein the setting element executes a linear movement into the housing element. If the release device additionally has a stamp for pressing in the fluid storage container, the stamp can be configured to execute a movement relative to a movement of the setting element. Thus, upon release of the fluid, the linear movement of the setting element can permit the pressing in of the fluid storage container according to the previously set fluid quantity.

The housing element can have an inwardly directed projection as a bearing surface of the spring. Thus, the spring can be protected in the housing element and can be held in position by the projection.

According to one embodiment, the spring can be mounted rotatably on the bearing surface of the projection. It is thus possible to prevent a situation where the spring is tensioned or untensioned during the rotation movement of the setting element when setting the fluid quantity that is to be released.

A method for producing a fluid discharge device for discharging a fluid comprises at least the following steps:
making available at least one component of the fluid discharge device with a release device for releasing the fluid from a fluid storage container, and with a force sensor which is configured to sense a restoring force acting on the release device upon release of the fluid; and
tensioning a spring between the release device and the force sensor, wherein the spring is configured to exert the restoring force on the force sensor during the release of the fluid.

This method can be executed by a variant of the proposed fluid discharge devices.

A method for operating a fluid discharge device for discharging a fluid comprises the following steps:
releasing the fluid from a fluid storage container; and
sensing a restoring force acting on a force sensor in the release step, wherein the restoring force is exerted by a spring tensioned between the force sensor and a release device.

This method can be implemented, for example, in software or hardware or in a combined form of software and hardware, for example in a control apparatus.

Also of advantage is a computer program product or computer program with program code, which can be stored on a machine-readable support or storage medium such as a semiconductor memory, a hard disk drive memory or an optical memory and which is used to carry out and/or control the steps of the method according to one of the embodiments described above, in particular when the program product or program is run on a computer or a device.

Illustrative embodiments of the approach presented here are shown in the drawings and are explained in more detail in the description below. In the drawings:

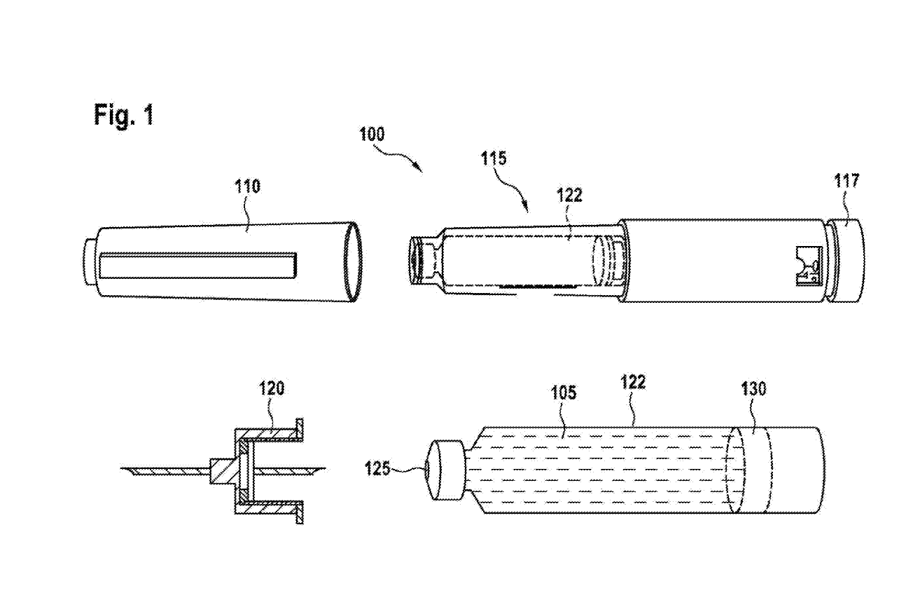

FIG. 1 shows a perspective view of a fluid discharge device;

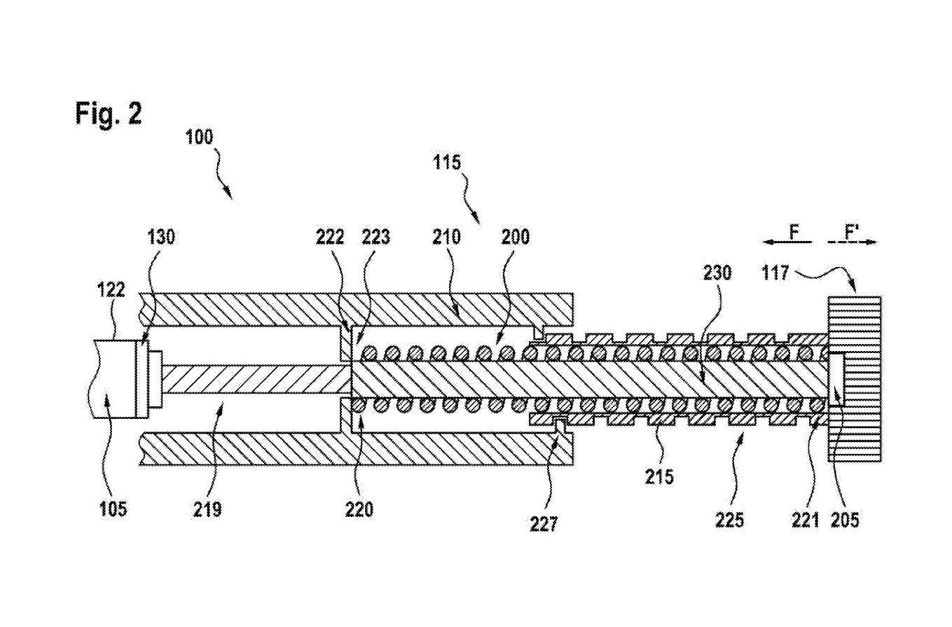

FIG. 2 shows a cross section of a component of a fluid discharge device according to an illustrative embodiment;

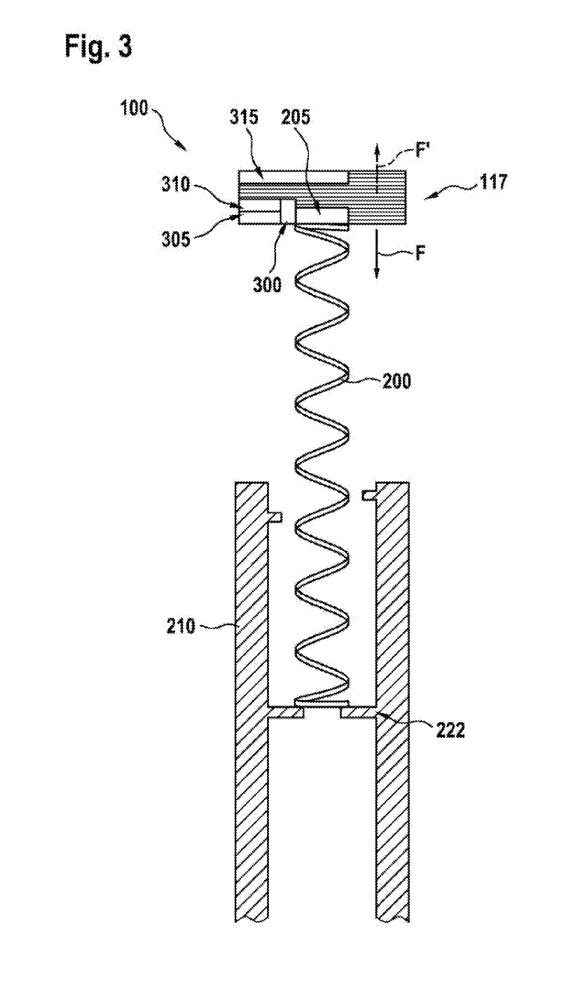

FIG. 3 shows a cross section of a component of a fluid discharge device according to an illustrative embodiment;

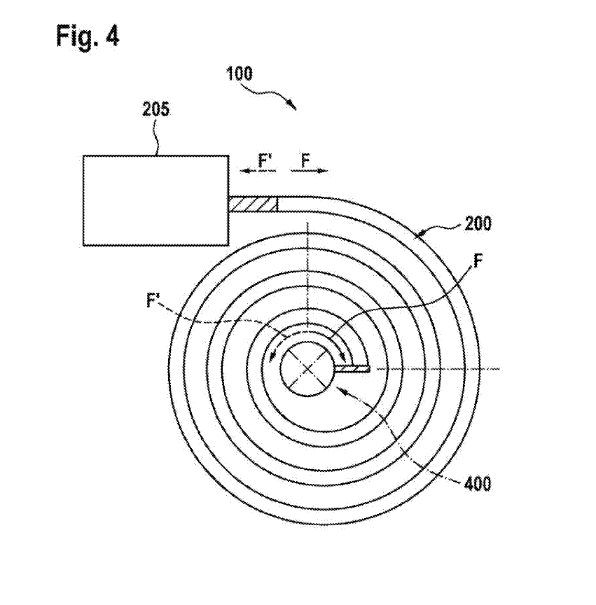

FIG. 4 shows a cross section of a component of a fluid discharge device according to an illustrative embodiment;

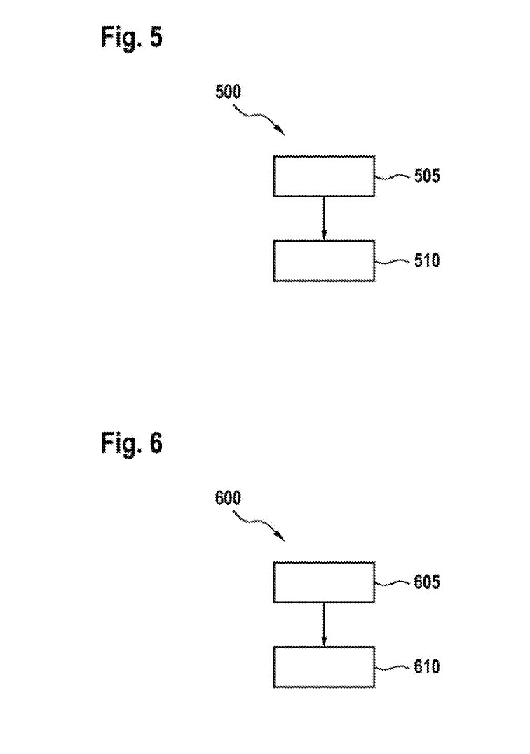

FIG. 5 shows a flow chart of a method for producing a fluid discharge device according to an illustrative embodiment; and

FIG. 6 shows a flow chart of a method for operating a fluid discharge device according to an illustrative embodiment.

In the following description of expedient illustrative embodiments of the current approach, the elements shown in the various figures and having similar effects are designated by the same or similar reference signs, thereby avoiding repeated description of these elements.

FIG. 1 shows a perspective view of a fluid discharge device 100 for discharging a fluid 105. The fluid discharge device 100 is designed as an insulin pen or an injection pen for injecting insulin 105. The fluid discharge device 100 has a cover 110, a release device 115, a dosing knob 117, and a needle 120.

In order to inject insulin 105, a fluid storage container 122 is first of all inserted into the release device 115, which fluid storage container 122 is configured as an insulin carpule which is filled with insulin 105 and may also be designated as an insulin cartridge. The fluid storage container 122 is a cylinder ampule which is provided at one end with a pierceable membrane 125. The opposite end of the fluid storage container 122 is closed with a movable stopper 130. When the fluid storage container 122 is received by the release device 115, the required quantity of insulin 105 is set by turning the dosing knob 117. The insulin 105 is subsequently released by pressing on the dosing knob 117.

FIG. 2 shows a cross section of a component of a fluid discharge device 100 according to an illustrative embodiment. This can be a component of the fluid discharge device 100 described with reference to FIG. 1 and having the release device 115, but with the difference that the fluid discharge device 100 here has a spring 200 and a force sensor 205. According to this illustrative embodiment, the fluid storage container 122 with the fluid 105 is received by the fluid discharge device 100. The release device 115 is configured to release the fluid 105 from the fluid storage container 122. For this purpose, the release device 115 optionally has a housing element 210, a setting element 215 and a stamp 219.

The spring 200 is tensioned between the release device 115 and the force sensor 205 and is configured to exert a restoring force F′ on the force sensor 205 upon release of the fluid 105. The force sensor 205 is configured to sense the restoring force F′ acting on the force sensor 205 upon release of the fluid 105.

According to this illustrative embodiment, the setting element 215 is partially received by the housing element 210. The spring 200 is enclosed from one side 220 in the housing element 210 and from an opposite, further side 221 by the setting element 215. The housing element 210 according to this illustrative embodiment has a projection 222 as a bearing surface 223 of the spring 200. According to this illustrative embodiment, the spring 200 is mounted rotatably on the bearing surface 223 of the projection 222. According to an alternative illustrative embodiment, the spring 200 is fixed non-rotatably on the bearing surface 223 (in this connection see FIG. 4). From the further side 221, the spring is fixed non-rotatably on the force sensor 205.

According to this illustrative embodiment, the force sensor 205 is arranged in the dosing knob 117. The dosing knob 117 is fixed at an end of the setting element 215, transversely with respect to a main extent of the setting element 215. To set a quantity of fluid to be released, the dosing knob 117 is turned, whereupon the setting element 215, in addition to executing the rotation movement, also executes a translation movement. The greater the rotation angle set, the farther the setting device in the form of the setting element 215 is rotated away from the projection 222 from an inner thread 227 of the housing element 210 via an outer thread 225. A distance between the dosing knob 117 and the housing element 210 then corresponds to the path length along which the fluid storage container 122 is emptied by means of a pressure exerted on the dosing knob 117, scaled with a transmission ratio factor. The stamp 219 presses on the stopper 130 of the fluid storage container 122. The stamp 219 has a threaded rod which can be rotated only in one direction via a locking mechanism. A mechanical component 230, designated as a driver, ensures that the dosing knob 117 can be unscrewed to the maximum extent for the remaining level of fluid 105 in the fluid storage container 120.

According to an alternative illustrative embodiment, the dosing knob 117 executes only the rotation movement during the setting of the fluid quantity, and the distance to the housing element 210 of the release device 115 is not changed. Here, as a result of the rotation movement and of the spring 200 fixed non-rotatably on the bearing surface 223, the spring 200 is tensioned, wherein the potential energy of the latter is converted by a mechanism to a translation movement of the stamp 219 during the release.

The restoring force F′ of the spring 200 is dependent on its elongation, i.e. length, in short s, and on a spring constant, in short D. The spring constant is dependent especially on the material of the spring 200 and the geometry of the latter, i.e. its wire diameter, spring diameter and number of turns. This relationship applies approximately both for the indicated restoring force F′ with a length change of the spring 200, and also a twisting of the spring 200 (shown in FIG. 4), when the spring 200 is a planar coil spring.

Details already discussed are explained in more detail below.

The dosing knob 117 is mounted on the end of a shaft in the form of the setting element, which has the outer thread 225. A mating piece of this outer thread 225 is arranged at the housing in the form of the housing element 210. The mechanical component 230, not considered in detail here, is arranged between the dosing knob 117 and a stationary structure in the form of the projection 222. The mechanical component 230 prevents a situation where a greater quantity of fluid can be set than is still present in the fluid storage container 122. The stamp 219 is in direct contact with the stopper 130 of the fluid storage container 122. The helical spring in the form of the spring 200 is connected fixedly to the dosing knob 117 at the further side 221. On the side 220, the spring 200 according to this illustrative embodiment is mechanically connected to the projection 222 in such a way that it too can be rotated during the setting of the fluid quantity. In an alternative illustrative embodiment described with reference to FIG. 4, the spring 200 is rigidly connected to the projection 222. According to this illustrative embodiment, the co-rotation of the spring 200 prevents a change in the number of turns of the spring 200, and, during the setting of the fluid quantity, there is only a change of length of the spring 200 in this illustrative embodiment.

The restoring force F′ of the spring 200 is measured with the force sensor 205. A mechanical connection to the spring 200 can be effected directly or via additional mechanical components.

FIG. 3 shows a cross section of a component of a fluid discharge device 100 according to an illustrative embodiment. The depicted features of the fluid discharge device 100 can be the features of the fluid discharge device 100 described with reference to FIG. 2. In addition, the fluid discharge device 100 according to this illustrative embodiment has a determination device 300, a time detection device 305, a communication interface 310, and a display device 315. The determination device 300, the time detection device 305, the communication interface 310, and the display device 315 are arranged in the dosing knob 117, for example.

The determination device 300 is configured to determine and store a released fluid quantity of the fluid, using the restoring force F′ sensed by the force sensor 205. The time detection device 305 is configured to detect and store at least one time point of the released fluid quantity. The display device 315 is configured to display at least the released fluid quantity and/or at least one stored fluid quantity and/or a time point of the stored fluid quantity. For this purpose, according to this illustrative embodiment, the display device 315 has an e-ink display. The communication interface 310 is configured to make at least the released fluid quantity available to an appliance arranged outside the fluid discharge device 100. According to this illustrative embodiment, the communication interface 300 is configured to make the released fluid quantity available to the appliance wirelessly. According to this illustrative embodiment, the fluid discharge device 100 moreover comprises an energy supply device, e.g. a button cell, which is configured to supply energy to the determination device 300, the time detection device 305, the communication interface 310, and the display device 315. Features, functions and advantages of the described fluid discharge device 100 are explained in more detail below.

Persons with diabetes often consider it a considerable benefit if, in an insulin pen in the form of the fluid discharge device 100, the last insulin dose in the form of the fluid quantity is continuously detected with the corresponding injection time. For example, compared to discharge devices that determine the fluid quantity by means of an optical method by having one or more light sources on one side of the fluid storage container and a CCD line camera for example on the other side of the fluid storage container, the proposed fluid discharge device 100 has the advantage that there is no sensitivity to contaminants in the optical path. Moreover, the proposed approach is much more cost-effective than, for example, discharge devices which use magnetic methods in which a ring magnet is rotated about a Hall sensor. Further advantages are that a method for operating the proposed fluid discharge device 100 is not based on an inductance change of the spring 200 when the latter changes in terms of its length, its cross section or its number of turns. Thus, there is no need here for the spring 200 to be contacted on both sides, which would signify considerable outlay for the return of the spring 200, and there is no need for a sliding contact, which wears mechanically and exhibits hysteresis.

The advantage of the approach proposed here is the provision of an injection appliance, in particular an insulin pen in the form of the fluid discharge device 100 with a spring 200 whose change of length or change in the number of turns exerts a restoring force F′ on the force sensor 205, of which the signal then indicates the dose setting in the form of the released fluid quantity. In addition to having the spring 200 and the force sensor 205, the fluid discharge device 100 also comprises a microcontroller for detection of measurement data in the form of the determination device 300 and electronic components in the form of the communication interface 310 for communication with external appliances, e.g. a smartphone, a trigger switch in the cap in the form of the dosing knob 117.

To put it another way: A sensor arrangement or dose sensor is proposed for detecting the actual injected insulin quantity with the spring 200 which either, as described with reference to FIGS. 2 and 3, is tensioned by its change of length, or, as described with reference to FIG. 4, is tensioned by a twisting or an increase of the number of turns and in so doing exerts a force on the force sensor 205.

The measurement of the restoring force F′ is effected upon activation of the injection, i.e. when pressure is applied to the dosing knob 117 in order to release the fluid. The restoring force F′ is then converted to the released fluid quantity, stored and shown on the integrated display in the form of the display device 315 and/or transmitted to an external appliance.

Advantages of the approach proposed here are:
increased user safety: a wrongly dosed quantity of insulin can have serious consequences in the short or long term,
less management effort since manual logging by the physician can be omitted and instead the automatically detected fluid quantity is electronically documented (e.g. in a smartphone app) and can be electronically transmitted,
very effective measuring method.

FIG. 4 shows a cross section of a component of a fluid discharge device 100 according to an illustrative embodiment. This can be one of the fluid discharge devices 100 described with reference to FIGS. 2 to 3, with the difference that the spring 200 according to this illustrative embodiment is fixed on the bearing face of the projection so as to be non-rotatable from the side described with reference to FIG. 2.

According to this illustrative embodiment, an increase of the fluid quantity caused by the rotation of the dosing knob 117 leads both to a change of length of the spring 200 and also to a change in the number of turns of the spring 200.

As was the case in FIG. 3, the restoring force F′ here is again all the higher the greater the quantity of fluid that is set. Since this relationship is bijective, a rotation angle can be determined immediately at the time the force sensor 205 is switched on. This is a huge advantage over incremental force sensors 205 for example, which, in order to produce a signal, always require a certain rotation angle to be scanned.

According to this illustrative embodiment, the force sensor 205 is quartz-based, piezo-based and MEMS-based. Reference sign 400 shows a rotation axis 400 of the dosing knob.

FIG. 5 shows a flow chart of a method 500 for producing a fluid discharge device according to an illustrative embodiment. This can be a method 500 for producing one of the fluid discharge devices described with reference to FIGS. 2 to 4. In a step 505, at least one component of the fluid discharge device, with a release device for releasing the fluid from a fluid storage container, and a force sensor, which is configured to sense a restoring force acting on the force sensor upon release of the fluid, are made available. In a tensioning step 510, a spring is tensioned between the release device and the force sensor, wherein the spring is configured to exert the restoring force on the force sensor upon release of the fluid.

FIG. 6 shows a flow chart of a method 600 for operating a fluid discharge device according to an illustrative embodiment. This can be a method 600 for operating one of the fluid discharge devices described with reference to FIGS. 2 to 4. In a release step 605, a fluid is released from a fluid storage container. In a sensing step 610, a restoring force acting on a force sensor in the release step 605 is detected, wherein the restoring force is exerted by a spring tensioned between the force sensor and a release device.

If an illustrative embodiment comprises an “and/or” link between a first feature and a second feature, this should be interpreted as meaning that the illustrative embodiment has both the first feature and the second feature in accordance with one embodiment and either only the first feature or only the second feature in accordance with a further embodiment.

1. A fluid discharge device for discharging a fluid, the fluid discharge device comprising:
a release device configured to release the fluid from a fluid storage container, the release device including a setting element configured to set a fluid quantity, and the release device including a housing element, the setting element inserted at least partially into the housing element upon release of the fluid;
a force sensor; and
a spring, tensioned between the release device and the force sensor, the spring configured to exert a restoring force on the force sensor upon release of the fluid, the spring is enclosed from one side in the housing element and from an opposite, further side by the setting element, wherein:
the force sensor, is arranged in a dosing knob at the further side,
the force sensor is configured to sense the restoring force acting on the force sensor upon release of the fluid, and
the spring is configured to be tensioned by a rotation movement of the dosing knob.

2. The fluid discharge device as claimed in claim 1, further comprising:
a determination device configured to use the sensed restoring force to determine and/or store a released fluid quantity of the fluid.

3. The fluid discharge device as claimed in claim 2, further comprising:
a time detection device configured to detect at least one time point of the released fluid quantity for storing the time point of the released fluid quantity.

4. The fluid discharge device as claimed in claim 2, further comprising:
a display device configured to display at least one of the released fluid quantity, at least one stored fluid quantity, and a time point of the stored fluid quantity.

5. The fluid discharge device as claimed in claim 2, further comprising:
a communication interface configured to make the released fluid quantity available to an appliance arranged externally with respect to the fluid discharge device.

6. The fluid discharge device as claimed in claim 1, wherein the force sensor is at least one of quartz-based, piezo-based, and MEMS-based.

7. The fluid discharge device as claimed in claim 1, wherein the fluid discharge device is configured to discharge the fluid as a medicament.

8. The fluid discharge device as claimed in claim 1, wherein:
the release device has a stamp for pressing in the fluid storage container, and
the stamp is configured to execute a movement relative to a movement of the setting element.

9. The fluid discharge device as claimed in claim 1, wherein the housing element has at least one projection as a bearing surface of the spring.

10. The fluid discharge device as claimed in claim 9, wherein the spring is mounted rotatably on the bearing surface of the projection.

11. A method for producing a fluid discharge device for discharging a fluid, the method comprising:
making available at least one component of the fluid discharge device with a release device for releasing the fluid from a fluid storage container, and with a force sensor which is configured to sense a restoring force acting on the release device upon release of the fluid, wherein the release device has a setting element for setting a fluid quantity, and a housing element, wherein the setting element is inserted at least partially into the housing element upon release of the fluid, and wherein the force sensor is arranged in a dosing knob at the further side; and
tensioning a spring between the release device and the force sensor, wherein the spring is configured to exert the restoring force on the force sensor upon release of the fluid, wherein the spring is tensioned in such a way that it is enclosed from one side in the housing element and from an opposite, further side by the setting element, and wherein the spring is further configured and arranged to be tensioned by a rotation movement of the dosing knob.

12. A computer program configured to execute the method as claimed in claim 11.

13. A machine-readable storage medium, on which the computer program as claimed in claim 12 is stored.

14. The fluid discharge device as claimed in claim 4, wherein the display device has an e-ink display.

15. The fluid discharge device as claimed in claim 5, wherein the communication interface is configured to make the released fluid quantity available wirelessly.

16. The fluid discharge device as claimed in claim 7, wherein the medicament is insulin.
